(19) United States
(12) Patent Application Publication
(10) Pub. No.: US 2012/0220990 A1
(43) Pub. Date: Aug. 30, 2012

(54) DISPOSABLE TIP WITH SHEATH

(75) Inventors: Richard James Mckenzie, Butler, PA (US); Lance Ranard Busch, Trafford, PA (US); Jason Paul Eaton, Hunker, PA (US)

(73) Assignee: KONINKLIJKE PHILIPS ELECTRONICS N.V., EINDHOVEN (NL)

(21) Appl. No.: 13/503,736

(22) PCT Filed: Oct. 4, 2010
(86) PCT No.: PCT/IB10/54473

§ 371 Date: Apr. 24, 2012

Related U.S. Application Data
(60) Provisional application No. 61/257,870, filed on Nov. 4, 2009.

(51) Int. Cl. A61B 17/00

(52) U.S. Cl. 606/1

(57) ABSTRACT

A disposable tip (20) having a sheath (30) and which is structured to be used with a medical device is provided. The sheath (30) provides protection for the medical device base unit (10). The sheath (30) is sealingly coupled to the tip (20), preferably at an end of the tip (20), proximal to the base unit (10). In this configuration, the sheath (30) may be extended to substantially enclose and protect the base unit (10) from contamination.

The invention relates to a disposable tip for a medical device and, more specifically, to a disposable tip having an integrated sheath.

Many medical devices include a base unit and a disposable patient interface. The base unit typically houses a meter or similar device while the patient interface is structured to be engaged by the patient. The disposable interface is typically elongated and extends away from the base unit. As such, the patient interface is hereinafter identified as a “tip.” This base unit, typically, has a coupling device or removable coupling structured to couple the tip to the base unit. The removable coupling may be, but is not limited to, a socket, into which the tip is removably inserted, or a protrusion, onto which a tip is removably attached. The removable coupling may be a threaded coupling. The removable coupling may provide for coupling beyond the physical coupling of the tip to the base unit. That is, if the base unit and tip utilize electronic or optical components, the removable coupling may provide for an electrical or optical connection.

An electronic thermometer, for example, has a base unit with a housing, a display, a processor or other programmable device, and a thermocouple or another heat measurement device. The housing acts as a handle or grip and encloses the electronics. The removable and disposable tip may include a heat conductor structured to conduct heat to the thermocouple in the base unit. The electronic thermometer tip is an elongated member structured to be inserted into the patient's mouth, ear, or other suitable location. In this example, the removable coupling would also provide a conductor interface structured to communicate heat from the tip to the thermocouple. As another example, the base unit may be structured to deliver ultraviolet UV-C light to reduce bacteria in a specific area of a patient's body. For this, the base unit may include an ultraviolet light and the tip may be a light pipe. In this instance, the removable coupling may include a passage, or an optical cable, structured to allow light from the base unit to be transmitted to the tip.

Such medical devices must be kept as sterile as possible so as to prevent cross-contamination between patients or between a patient and the user of the device. Thus, the tip, i.e. the patent interface, is typically disposable. This allows for a new, sterile tip to be attached to the base unit prior to use with a new patient. This does not, however, prevent indirect contamination of the base unit. For example, a patent may sneeze while the tip is inserted within their mouth. Cast off saliva, or other fluids, could contaminate the base unit. Sterilizing the base unit is, typically, costly and time consuming.

To protect the base unit, medical personnel are known to use a protective barrier, typically a plastic bag or a similar device. That is, users are known to place the entire device, base unit and tip, within a plastic bag during use. While this quick fix provides some protection for the base unit, the bag may interfere with the operation of the device. The bag may also be uncomfortable for the patient. That is, tips are typically shaped to correspond to the area in which the tip is used. When a bag is placed over the tip, the ergonomic form of the tip is disturbed. Further, users may become complacent with tip removal. That is, if the user believes that the tip has been protected from contamination by the bag, the user may not replace the tip prior to each use. This contravenes the standard practice of providing a new, sterile tip for each patient.

The concept disclosed and claimed herein provides for a disposable tip that incorporates a sheath. The sheath provides protection for the base unit. The sheath is sealingly coupled to the tip, preferably at an end of the tip proximal to the base unit. In this configuration, the sheath may be extended to substantially enclose and protect the base unit from contamination. Further, the tip is unobstructed and, as such, there is nothing to interfere with the operation of the tip or the comfort of the patient.

These and other features and characteristics of the present concept, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural references unless the context clearly dictates otherwise.

A full understanding of the invention can be gained from the following description of the illustrated embodiments when read in conjunction with the accompanying drawings in which:

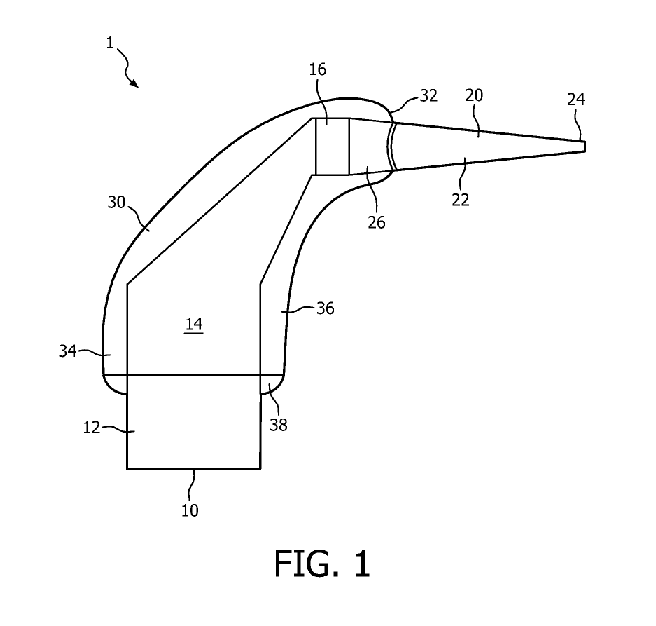

FIG. 1 is a schematic side view of a medical device with a removable tip.

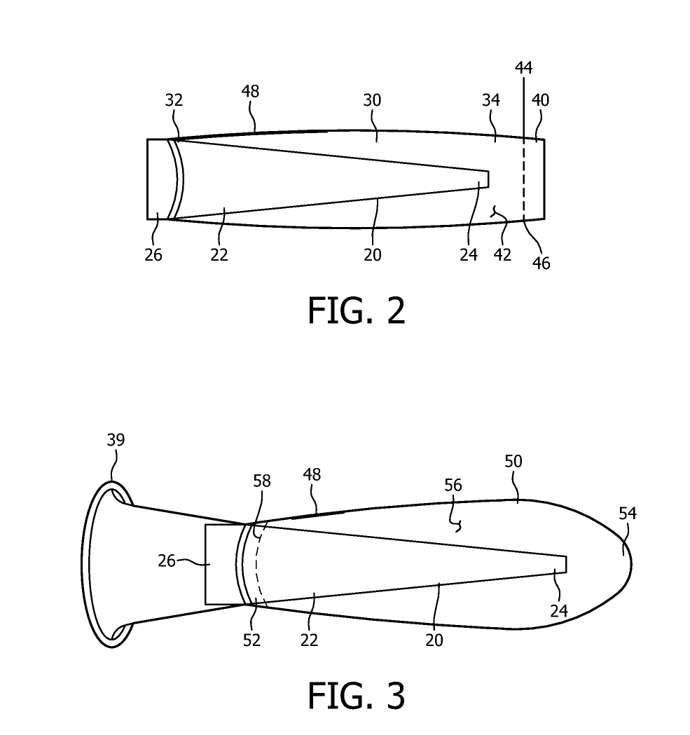

FIG. 2 is schematic side view of an alternate embodiment of the tip with a sheath.

FIG. 3 is schematic side view of an alternate embodiment of the tip with a sheath.

As used herein, “coupled” means a link between two or more elements, whether direct or indirect, so long as a link occurs.

As used herein, “directly coupled” means that two elements are directly in contact with each other.

As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body.

As used herein, “adjacent” means at or near.

The background set forth above and the description set forth below utilize the convention of a medical device used by medical personnel on a patient. While this is a typical situation, the claims are not so limited. For example, it is understood that the claims are applicable to disposable tips for base units wherein the patient uses the medical device on themselves, or, where a parent uses the medical device on a child, where the device is used on an animal, etc.

As shown in FIG. 1, a medical device 1 includes a base unit 10 and a disposable tip 20. The base unit 10 includes a housing 12 that may act as a handle 14. Operational components of the medical device 1, e.g. electronics, controls, output devices (typically displays and/or speakers), which are not relevant to the present discussion are typically disposed on or within the housing 12. The base unit 10 has a removable coupling 16 structured to removably engage the tip 20. The removable coupling 16 may be, but is not limited to, a socket (not shown), into which the tip 20 is removably inserted, or a protrusion (not shown), onto which the tip 20 is removably attached. The removable coupling 16 may be a threaded coupling. The removable coupling 16 may provide for coupling beyond the physical coupling of the tip 20 to the base unit 10. That is, for example, if the base unit 10 and tip 20 utilize electronic components, the removable coupling 16 may provide for an electrical connection.

The tip 20 includes a body 22 and a sheath 30. The body 22 has a distal end 24 and a proximal end 26. The body proximal end 26 is structured to be coupled to the base unit removable coupling 16. The shape of the body 22 corresponds to the purpose of the medical device 1 and typically selected from the group including: a light pipe, a swab, a brush, a sensor, an optical lens, or a tool configured for a specific surgery or procedure. Further, the shape is typically an elongated body 22 wherein the longitudinal axis of the body aligns with the center of the removable coupling 16.

The sheath 30 is thin, flexible, and generally tubular. As the sheath 30 may cover the base unit 10 controls, the sheath 30 is, preferably, transparent. The sheath 30 is preferably made from a plastic or elastomeric material and, more preferably, latex. The sheath 30 has a first end 32, an elongated medial portion 33, and a second end 34. The sheath 30 also has a first surface 36 and a second surface 38. It is noted that, as the sheath 30 is reversible (moves between two configurations, as described below) either the sheath first surface 36 or the sheath second surface 38 may be the “inner” surface or the “outer surface.” The sheath 30 is sealingly coupled to the body 20. The sheath 30 may be sealingly coupled to the body 20 by any coupling means such as, but not limited to, a coupling means selected from the group including: heat bonding, adhesives, ultrasonic welding and captive assembly. Preferably, the sheath first end 32 is coupled adjacent to the body proximal end 26.

The sheath 30 is structured to substantially protect the base unit 10 during use of the medical device 1. Thus, the sheath 30 is sized to substantially enclose at least the base unit removable coupling 16. More preferably, the sheath 30 is sized to substantially cover the base unit 10. The sheath 30 is movable between two positions, a storage position and a use position. In the storage position, the sheath 30 may be used to protect the tip body 22 or may simply be in a rolled configuration. Both such embodiments are discussed below.

In one embodiment, the sheath 30 is used to protect the tip body 22 prior to use. That is, the sheath 30 is structured to move between a first position, wherein the sheath 30 substantially encloses the body 22, and a second position wherein the sheath 30 does not substantially enclose the body 22. When the sheath 30 is not used to enclose the body 22, the sheath 30 may be disposed over the base unit 10.

As the tip 20 may be required to be sterile, the sheath 30 may be used as a protective barrier. That is, the sheath second end 34 may have a removable dam 40 sealingly coupled thereto. In this configuration, the sheath 30 defines an enclosed space 42. Thus, when the sheath 30 is in the first position, the body 22 is disposed within the enclosed space 42. Preferably, the sheath 30 and the dam 40 are portions of a body formed as a unitary component. In this configuration, the sheath 30 and the dam 40 have a similar thickness. The sheath 30 and the dam 40 meet at an interface 44 separating the portions of the unitary body. To aid in removal of the dam 40, the sheath/dam interface 44 has a portion 46 with a reduced thickness. The portion 46 with a reduced thickness may be formed by any means such as, but not limited to, scoring the material at the sheath/dam interface 44 or molding the unitary body with a reduced thickness at the sheath/dam interface 44. The portion 46 may be intermittent, similar to perforations but not extending through the unitary body, or may extend over all of the sheath/dam interface 44. In this configuration, the user may easily separate the sheath 30 and the dam 40.

Further, in this configuration, the sheath 30 may be used to further enhance the protection of the body 22 through use of a coating 48. That is, when the sheath 30 is in the first position, the inner side may have coating 46 selected from the group including: saline and an antibacterial agent in a liquid, gel or paste.

As noted above, the size of the sheath 30 typically is based on the size of the base unit 10. As such, the sheath 30 may be too large to enclose the body 22 in a compact configuration. Accordingly, the sheath 30 may also include a tip pocket 50. The tip pocket 50 is an extension from the sheath first end 32. The tip pocket 50 is also a thin, flexible, generally tubular member having a first end 52 and a second end 54. The tip pocket first end 52 is sealingly and removably coupled to the sheath first end 32. The tip pocket second end 54 is closed. Thus, the tip pocket 50 defines an enclosed space 56. The body 22 is disposed in the tip pocket enclosed space 56. The tip pocket 50 may be formed as a unitary body with the sheath 30. As with the dam 40, the sheath 30 and the tip pocket 50, preferably, have the same thickness and may be identified as portions of a unitary body that meet at an interface 58. Thus, as before, the removal of the tip pocket 50 may be made easier by having a reduced thickness over a portion of, or all of, the sheath/tip pocket interface 58. Further, in a similar manner as described above, the tip pocket 50 may be used further enhance the protection of the body 22 through use of a coating 48. That is, when the sheath 30 is in the first position, the inner side may have coating 48 selected from the group including: saline and an antibacterial agent in a liquid, gel or paste.

In another embodiment, which may be used with the tip pocket 50, the sheath 30 is reduced to a roll 39 (discussed below) about the body 22. That is, when the sheath 30 has an open second end 34, the second end 34 may be rolled over the sheath first surface 36 extending over substantially all of the elongated sheath medial portion 33. In this first, rolled configuration the elongated medial portion 33 is rolled, generally snugly, about the second end 34 thereby creating a roll 39. This configuration is similar to the rolled configuration of a condom and the sheath 30 may be unrolled in a similar manner. That is, in a second, unrolled configuration, the medial portion 33 is substantially unrolled from the second end 34. This action may be performed after the tip 20 is coupled to the base unit 10.

Although the concept has been described in detail for the purpose of illustration based on what is currently considered to be the most practical and illustrated embodiments, it is to be understood that such detail is solely for that purpose and that the concept is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A disposable tip (20) for use with a base unit (10), the base unit (10) having a removable coupling (16) structured to removably engage the tip (20), the tip (20) comprising:
a body (22) having a distal end (24) and a proximal end (26); and
a thin, flexible, generally tubular sheath (30) sealingly coupled to the body (22), the sheath (30) sized to substantially enclose at least the base unit removable coupling (16).

2. The disposable tip (20) of claim 1 wherein:
the sheath (30) has a first end (32) and a second end (34);
the sheath first end (32) coupled adjacent to the body proximal end (26); and
the sheath (30) is movable between two positions, a first position, wherein the sheath (30) substantially encloses the body (22), and a second position wherein the sheath (30) does not substantially enclose the body (22).

3. The disposable tip (20) of claim 2 wherein:
the sheath second end (34) having a removable dam (40), the removable dam (40) sealingly coupled to the sheath second end (34), thereby defining an enclosed space (42);
wherein, when the sheath (30) is in the first position, the body (22) is disposed within the enclosed space (42); and
wherein the removable dam (40) must be removed prior to moving the sheath (30) into the second position.

4. The disposable tip (20) of claim 3 wherein the dam (40) is formed as a unitary body (22) with the sheath (30).

5. The disposable tip (20) of claim 4 wherein;
the sheath (30) and the dam (40) have about the same thickness;
the sheath (30) and the dam (40) are coupled at an interface (58); and
the sheath/dam interface (58) having at least a portion (46) with a reduced thickness.

6. The disposable tip (20) of claim 3 wherein, when the sheath (30) is in the first position, the inner side of the sheath (30) includes a coating (48).

7. The disposable tip (20) of claim 6 wherein the coating (48) is selected from the group including: saline and an antibacterial agent in a liquid, gel or paste.

8. The disposable tip (20) of claim 2 wherein:
the sheath (30) includes a tip pocket (50) structured to enclose the body (22);
the tip pocket (50) being a thin, flexible, generally tubular member having a first end (52)and a second end (54);
the tip pocket first end (52) sealingly and removably coupled to the sheath first end (32);
the tip pocket second end (54) being closed, thereby defining an enclosed space (56); and
the body (22) disposed in the tip pocket enclosed space (56).

9. The disposable tip (20) of claim 8 wherein the tip pocket (50) is formed as a unitary body (22) with the sheath (30).

10. The disposable tip (20) of claim 8 wherein:
the sheath (30) and the tip pocket (50) have about the same thickness;
the sheath (30) and the tip pocket (50) are coupled at an interface (58); and
the sheath/tip pocket interface (58) having at least a portion (46) with a reduced thickness.

11. The disposable tip (20) of claim 8 wherein the inner side of the tip pocket (20) includes a coating (48).

12. The disposable tip (20) of claim 11 wherein the coating (48) is selected from the group including: saline and an antibacterial agent in a liquid, gel or paste.

13. The disposable tip (20) of claim 1 wherein:
the sheath (30) has a first end (32) and a second end (34), and, a first surface (36) and a second surface (38);
the sheath first end (32) is coupled to the body proximal end (26);
the sheath second end (34) being open;
the sheath (30) having an elongated medial portion (33) between the sheath first end (32) and the sheath second end (34);
the sheath (30) structured to be reconfigured from a first rolled configuration, wherein the second end (34) is rolled over the first surface (36) of substantially all of the elongated medial portion (33), to a second unrolled configuration, wherein the elongated medial portion (33) is substantially unrolled from the second end (34); and
wherein, when the body (22) is in the first rolled configuration, the elongated medial portion (33) is rolled, generally snugly, about the second end (34) thereby creating a roll (39).

14. The disposable tip (20) of claim 1 wherein the sheath first end (32) is coupled to the body (22) adjacent the body proximal end (26).

15. The disposable tip (20) of claim 1 wherein the sheath (30) is sized to substantially cover the base unit (10).

16. The disposable tip (20) of claim 1 wherein the sheath (30) is substantially transparent.

17. The disposable tip (20) of claim 1 wherein the body (22) is selected from the group including: a light pipe, a swab, a brush, a sensor, an optical lens, a tool configured for a specific surgery or procedure.

18. The disposable tip (20) of claim 1 wherein the body (22) is elongated.

19. The disposable tip (20) of claim 1 wherein the sheath (30) is sealingly coupled to the body (22) by a coupling means selected from the group including: heat bonding, adhesives, and ultrasonic welding and captive assembly.
